(19) United States
(12) Patent Application Publication
(10) Pub. No.: US 2023/0365626 A1
(43) Pub. Date: Nov. 16, 2023

(54) Alloferon Peptide and Method Using the Same

(71) Applicants: Industry Academy Cooperation Foundation of Sejong University, Seoul (KR); University of Ulsan Foundation for Industry Cooperation, Ulsan (KR); The Asan Foundation, Seoul (KR)

(72) Inventors: Yang-Hee KIM, Seoul (KR); Ki Ryeong KIM, Incheon (KR); Dongyeol LIM, Seoul (KR); Hyung Wook KIM, Gyeonggi-do (KR); Jae-Young KOH, Seoul (KR)

(21) Appl. No.: 18/325,582

(22) Filed: May 30, 2023

Related U.S. Application Data
(63) Continuation of application No. PCT/KR2021/017800, filed on Nov. 29, 2021.

(30) Foreign Application Priority Data
Nov. 30, 2020 (KR) 10-2020-0165032

(51) Int. Cl. C07K 7/08; A61P 25/28

(52) U.S. Cl. CPC C07K 7/08; A61P 25/28; A61K 38/00

(57) ABSTRACT

The present disclosure provides a method for treating a degenerative neuronal disease, comprising administering, to an individual suffering from neurodegenerative disease, a therapeutically effective amount of an alloferon peptide having an amino acid sequence of SEQ ID NO: 1.

### CROSS-REFERENCE TO RELATED APPLICATION

This application claims priority to Korean Patent Application No. 10-2020-0165032, filed on Nov. 30, 2020, and International Patent Application No. PCT/KR2021/017800, filed on Nov. 29, 2021, and all the benefits accruing therefrom under 35 U.S.C. § 119, the contents of which are incorporated by reference in their entirety.

### REFERENCE TO SEQUENCE LISTING SUBMITTED ELECTRONICALLY

The content of the electronically submitted sequence listing in ST26 file (Name: 52520001US01SEQLXML.xml; Size: 6 KB; and Date of Creation: May 30, 2023) filed with the application is incorporated herein by reference in its entirety.

### BACKGROUND

The present disclosure relates to a novel alloferon peptide and a method using the same, and more specifically, to a method for treating degenerative neuronal diseases using the novel alloferon peptide.

Zinc is a trace element essential for cell proliferation or differentiation and is known as a cofactor involved in functions and structures of proteins such as enzymes or transcription factors. In addition, it is known that zinc homeostasis in nerve cells plays a key role in the survival of nerve cells, and when zinc in neurons is deficient, apoptosis is induced, which causes degenerative neuronal diseases such as Alzheimer's disease (AD) and Parkinson's disease ((Lien, H. et al., BBRC. 268: 148-154, 2000), and in contrast, when excess zinc is present in nerve cells, it is also known that cell damage is induced, which may lead to acute brain injuries such as ischemia or seizure (Koh et al., Science. 272: 1013-1016, 1996).

Meanwhile, autophagy is an intracellular mechanism for obtaining an energy source by decomposing cell organelles in a starvation situation or removing damaged cell organelles or abnormal or pathological protein aggregates. During autophagy, the components of the cytoplasm are surrounded by a double membrane and isolated from other cell organelles to form an autophagosome. In this case, soluble light chain I (LC3I) present in the cytoplasm is converted to the form of light chain II (LC3II) bound to the autophagosomal membrane. The autophagosome is then fused with lysosome to form autolysosome and is degraded and recycled by several hydrolytic enzymes present in the lysosome.

Recently, it has been known that aggregates of proteins, such as α-synuclein or β-amyloid peptides, appearing in degenerative neuronal diseases may be removed when autophagy is promoted. In fact, studies have shown that when there is a defect in the function of autolysosomes, the degradation of these protein aggregates is inhibited, so that the flux of autophagy is blocked, and eventually, the absence of such autophagy results in the accumulation of by-products of degenerative neuronal diseases (Zhang et al., ABBS. 41(6): 437-445, 2009; Lee et al., Cell. 141(7): 1146-1158, 2010).

Previous studies have shown that the function of lysosome may be improved by supplying zinc, and there are findings showing that treating, with TPEN, which is a strong zinc chelator, lysosomal membrane permeabilization (LMP) induced with H₂O₂, tamoxifen, and ethanol inhibits lysosomal membrane permeabilization phenomenon, and conversely, when the autophagy is reduced, supplying additional zinc may promote autophagy (Lee et al., Glia 57: 1351-1361, 2009; Hwang et al., Biometals 23: 997-1013, 2010; Liuzzi et al., Biol. Trace Elem. Res. 156: 350-356, 2013), and thus zinc is assumed to have an important influence on autophagy. However, no drugs have been developed that enables the treatment of degenerative neuronal diseases by maintaining zinc homeostasis in nerve cells.

### SUMMARY

The present disclosure provides a novel alloferon peptide capable of treating degenerative neuronal diseases by maintaining zinc homeostasis in nerve cells, thereby removing abnormal or pathological protein aggregates such as amyloid beta peptide and tau proteins, which are pathological substances of degenerative neuronal diseases, through autophagy or lysosome function improvement, and a method using the novel alloferon peptide.

According to one aspect of the present disclosure, there is provided a method for treating neurodegenerative disease, comprising administering, to an individual suffering from a degenerative neuronal disease, a therapeutically effective amount of an alloferon peptide having an amino acid sequence of SEQ ID NO: 1.

According to another aspect of the present disclosure, there is provided a modified alloferon peptide in which at least one amino acid of an alloferon peptide having an amino acid sequence of SEQ ID NO: 1 is substituted with a D-form amino acid.

According to still another aspect of the present disclosure, there is provided a composition containing the modified alloferon peptide as an active ingredient.

According to yet another aspect of the present disclosure, there is provided a method for treating neurodegenerative disease, comprising administering, to an individual, a therapeutically effective amount of the modified alloferon peptide.

### BRIEF DESCRIPTION OF THE DRAWINGS

Exemplary embodiments can be understood in more detail from the following description taken in conjunction with the accompanying drawings, in which:

FIG. 1 shows the results of examining whether alloferon L-form peptides and D-form peptides are decomposed in the plasma of mice, and the results are obtained by treating separate plasma with 10 μM of each of the L-form and D-form alloferon peptides, reacting each for 0, 10, 30, 60, and 90 minutes at 37° C., and then measuring the quantitative change through mass spectrometry.

FIG. 2A shows a result showing that the autophagic flux blocked by chloroquine is recovered by alloferon D-form peptides, and is a fluorescence microscopic image obtained by taking LC3-GFP puncta 6 hours after an H4 cell line (GL-H4) in which GFP-LC3 proteins were stably overexpressed, was treated with the indicated concentrations of alloferon D-form peptides alone or in combination with chloroquine, FIG. 2B is a graph showing the results of the quantification of LC3-GFP puncta size 6 hours after the GL-H4 cell line was treated with the indicated concentrations of alloferon D-form peptides alone or in combination with chloroquine, and FIG. 2C is a photograph showing the result of western blot analysis in which the expression levels of p62 and LC3 proteins were measured for protein samples obtained 6 hours after cortical nerve cells were treated with chloroquine alone or in combination with the indicated concentrations of alloferon D-form peptides.

FIGS. 3A and 3B show experimental results showing changes in lysosomal pH according to the treatment of alloferon D-form peptides, FIG. 3A shows a fluorescence microscopic image showing the results of staining with lysotracker 4 hours after the cortical nerve cells were respectively treated with the indicated concentrations of alloferon D-form peptides alone and a graph of quantification of the fluorescence intensity, and FIG. 3B shows a fluorescence microscopic image showing the results of staining with lysotracker 4 hours after the cortical nerve cells were treated with 20 μM alloferon D-form peptides alone, 25 μM zinc alone, 50 μM chloroquine alone, 20 μM alloferon D-form+50 μM chloroquine, 25 μM zinc+50 μM chloroquine, respectively, and a graph of quantification of the fluorescence intensity.

FIGS. 4A and 4B show experimental results showing changes in the enzymatic activity of cathepsin B according to the treatment of alloferon D-form peptides, FIG. 4A shows a fluorescence microscopic image showing the assay results of the enzymatic activity of cathepsin B in cells with in situ Cathepsin B activity kit (Magic Red Cathepsin B Detection Kit) 1 hour after the cortical nerve cells are treated with 2 μM alloferon D-form peptides alone and 20 μM alloferon D-form peptides alone, respectively, and a graph of quantification of fluorescence intensity, and FIG. 4B shows a fluorescence microscopic image showing the assay results of the enzymatic activity of cathepsin B in cells with in situ Cathepsin B activity kit 4 hours after the cortical nerve cells were treated with 2 μM alloferon D-form peptides alone and 20 μM alloferon D-form peptides alone, respectively, and a graph of quantification of fluorescence intensity.

FIGS. 5A-5C show the results of examining the level of lysosomal biosynthesis and protease expression according to the treatment of alloferon D-form peptides, FIG. 5A shows western blot analysis results of measuring the expression levels of TFEB, cathepsin B, and cathepsin D on protein samples obtained 6 hours after the cortical nerve cells were treated with the indicated concentrations of alloferon D-form peptides, FIG. 5B shows western blot analysis results of measuring the expression levels of TFEB, cathepsin B, and cathepsin D on protein samples obtained at indicated times after the cortical nerve cells were treated with 20 μM alloferon D-form peptides alone or 4 hours after the cortical nerve cells were treated with 20 μM alloferon D-form peptide+1 μM TPEN, and 1 μM TPEN, and FIG. 5C shows a fluorescence microscopic image showing the results obtained by observing the positions after the cortical nerve cells were treated with 20 μM alloferon D-form peptides alone for the indicated times, respectively, and immunostaining is performed with TFEB antibodies.

FIG. 6 shows experimental results showing changes in the concentration of free zinc in the lysosome according to treatment of alloferon D-form peptides, and is a fluorescence microscopic image showing the results of staining with Zinpyr-1 and lysotracker 1 hour after the cortical nerve cells were treated with 2 μM alloferon D-form peptides alone and 20 μM alloferon D-form peptides alone, respectively, and 4 hours after the cortical nerve cells were treated with 2 μM alloferon D-form peptides alone, 20 μM alloferon D-form peptides alone, and in combination with 1 μM TPEN.

FIGS. 7A and 7B show the results of observing whether beta-amyloid 1-42 (Aβ₁₋₄₂) protein aggregates are degraded by the treatment of alloferon D-form peptides, FIG. 7A shows a fluorescence microscopic image obtained by taking fluorescence generated by the beta-amyloid 1-42 protein aggregates 12 hours after 293T cells transfected so as to express the beta-amyloid 1-42 were treated with 100 nM bafilomycin A1 alone, and 100 nM bafilomycin A1 in combination with the indicated concentrations (2 or 20 μM) of alloferon D-form peptides or 25 μM zinc and a graph showing the results of measuring relative fluorescence intensity, and FIG. 7B shows a photograph showing the result of western blot analysis in which the expression level of the beta-amyloid protein for the protein sample obtained 12 hours after 293T cells transfected so as to express the beta-amyloid 1-42 (Aβ₁₋₄₂) were treated with 100 nM bafilomycin A1 alone or 100 nM bafilomycin A1 in combination with the indicated concentrations of alloferon D-form peptides or 25 μM zinc, and a graph showing the quantification results of relative expression levels.

FIGS. 8A and 8B show the results of observing whether mutant Huntington protein aggregates are degraded by the treatment of alloferon D-form peptides. FIG. 8A shows a fluorescence microscopic image obtained by taking fluorescence generated by the mutant Huntington protein aggregates 12 hours after 293T cells transfected so as to express the mutant Huntington protein (mt-Htt) were treated with 100 nM bafilomycin A1 alone, and 100 nM bafilomycin A1 in combination with 20 μM alloferon D-form peptides or 25 μM zinc and a graph showing the results of measuring relative fluorescence intensity, and FIG. 8B shows a photograph showing the result of western blot analysis in which the expression level of the Huntington protein for the protein sample obtained 12 hours after the 293T cells transfected so as to express the mutant Huntington protein were treated with 100 nM bafilomycin A1 alone or 100 nM bafilomycin A1 in combination with 20 μM alloferon D-form peptides or 25 μM zinc, and a graph showing the quantification results of relative expression levels.

FIGS. 9A and 9B show the results of observing whether alloferon D-form peptides are introduced by endocytosis, shows the result of western blot analysis of measuring the expression levels of p62 and LC3 proteins for the protein sample obtained 12 hours after the cortical nerve cells were treated with 100 nM bafilomycin A1 alone, 100 nM bafilomycin A1+20 μM alloferon D-form peptides, and 100 nM bafilomycin A1+20 μM alloferon D-form peptides+LRP-1 antibodies, respectively, and shows the result of western blot analysis of measuring the expression levels of p62 and LC3 proteins for the protein sample obtained 12 hours after the cortical nerve cells were treated with 100 nM bafilomycin A1 alone, 100 nM bafilomycin A1+20 μM alloferon D-form peptides, 100 nM bafilomycin A1+20 μM alloferon D-form peptides+500 μM methyl-beta-cyclodextrin, and 100 nM bafilomycin A1+20 μM alloferon D-form peptides+500 μM methyl-beta-cyclodextrin+2 μM chloropromazine, respectively.

FIGS. 10A-10C show the results of confirming changes in immune response and body weight after injection of alloferon D-form peptides into the body of mice, FIG. 10A shows the results of enzyme-linked immunosorbent assay of measuring changes in IL-6 and TNF-α in plasma obtained from blood collected 1 hour after the mice were intraperitoneally injected with the indicated concentrations of alloferon D-form peptides, and results of measuring changes in body weight during the intraperitoneal injection of 16 mg/kg of alloferon D-form peptides for 14 weeks.

FIG. 11 shows the results of testing the memory of mice injected with alloferon D-form peptides and is a graph showing the average value in which the mice in a control or a dementia model injected with saline or alloferon D-form peptides were acclimated and undergo a water maze test four times every day for 5 days and the time for the mice to find an escape platform was measured every day.

FIG. 12A shows the results of confirming changes in β-amyloid accumulation and autophagic flux in brains of mice injected with alloferon D-form peptides, and is a photograph showing the result of western blot analysis performed using antibody 6E10 capable of specifically detecting the expression level of beta-amyloid after obtaining protein samples from the cerebral cortex and hippocampus obtained from the brains of mice in a control or a dementia model whose behavioral experiment had been completed, FIG. 12B shows a fluorescence microscopic image (top) showing changes in amyloid plaques through Thioflavin S staining in the brain slices obtained from the brains of the mice in the control or the dementia model, and a graph (bottom) of quantification of the number of the amyloid plaques observed in the fluorescence microscopic image, and FIG. 12C shows a photograph (top) showing the result of western blot analysis of measuring the expression levels of p62 and LC3 proteins by obtaining protein samples from the cerebral cortex and hippocampus obtained from the brains of the mice in the control or the dementia model and a graph (bottom) of quantification of the expression levels.

### DETAILED DESCRIPTION OF EMBODIMENTS

Hereinafter, specific embodiments will be described in detail with reference to the accompanying drawings.

### Definition of Terms

As used herein, the term “zinc homeostasis” refers to a mechanism for maintaining the concentration of zinc in a cell at a certain level, and it is known that zinc transporters, zinc-binding proteins (metallothioneins; MTs), transcription factor (MTF1-2), and the like are involved in maintaining the concentration of zinc in a cell at a certain level.

As used herein, the term “degenerative neuronal disease” refers to a disease characterized by the progressive loss of structure or function of the nerve due to the abnormal death of nerve cells. These degenerative neuronal diseases include amyotrophic lateral sclerosis (ALS), Parkinson's disease (PD), Alzheimer's disease (AD), Huntington's disease (HD), and the like.

As used herein, the term “abnormal or pathological protein aggregates” means that proteins such as amyloid or tau are abnormally aggregated in cells to form insoluble fibrills. These abnormal or pathological protein aggregates are known as neuropathological features of various intermittent or genetic degenerative neuronal diseases.

As used herein, the term “alloferon” refers to a natural peptide consisting of 13 amino acids separated from the larval blood of bacteria-infected Caliphora vicina insect, which is a non-toxic antiviral agent (Korean Patent No. 394864) developed to treat infections with viruses such as influenza viruses and herpes viruses, and has four histidine groups capable of binding to metal ions including zinc.

### DETAILED DESCRIPTION

According to one aspect of the present disclosure, there is provided a method for treating a neurodegenerative disease, comprising administering, to an individual suffering from a neurodegenerative disease, a therapeutically effective amount of an alloferon peptide having an amino acid sequence of SEQ ID NO: 1.

In the treatment method, the alloferon peptide may have typical L-form amino acids, including at least one D-form amino acid, or have amino acids, all of which are substituted with D-form amino acids.

In the treatment method, the degenerative neuronal disease may have the formation of abnormal protein aggregates as a cause of the disease or a pathological phenomenon, the abnormal protein aggregates may be formed by abnormal aggregation of α-synuclein, β-amyloid, Huntington protein, or tau protein, and the degenerative neuronal disease having the formation of abnormal protein aggregates as a cause of the disease or a pathological phenomenon may be Alzheimer's disease (AD), Parkinson's disease (PD), Huntington's disease (HD), chronic traumatic encephalopathy, Lytico-bodig disease, temporal lobe degeneration, corticobasal degeneration, progressive supranuclear palsy, or ganglioglioma.

In the treatment method, the alloferon peptide may treat neurodegenerative disease by preventing cell death of nerve cells and regulating zinc homeostasis in cells.

According to another aspect of the present disclosure, there is provided a modified alloferon peptide in which at least one amino acid of an alloferon peptide having an amino acid sequence of SEQ ID NO: 1 is substituted with a D-form amino acid.

In the peptide, the at least one amino acid may be a histidine residue, all four histidines may be substituted with D-form histidines, and all of the amino acids may be substituted with D-form amino acids.

In the peptide, all the amino acids except for the four histidines may be substituted with D-form amino acids, all the amino acids except for the third and fourth amino acids may be substituted with D-form amino acids, and the third and fourth amino acids may be substituted with D-form amino acids.

According to still another aspect of the present disclosure, there is provided a composition containing the modified alloferon peptide as an active ingredient.

According to yet another aspect of the present disclosure, there is provided a method for treating a degenerative neuronal disease, comprising administering, to an individual, a therapeutically effective amount of the modified alloferon peptide.

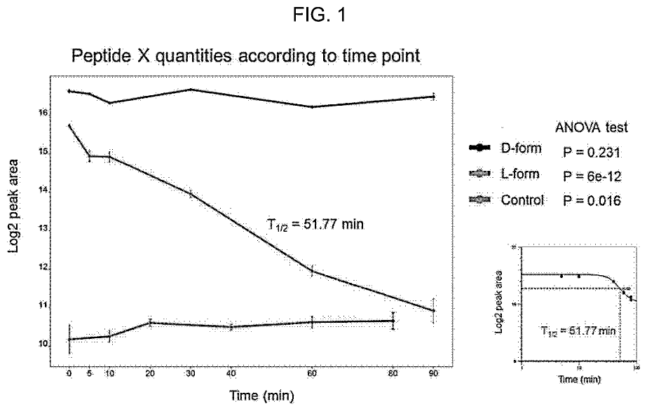

As shown in FIG. 1 of the present disclosure, the present inventors have found that L-form alloferon naturally present in the body showed excellent activity under in vitro conditions, but it is not easy to develop the L-form alloferon into an actual medicine due to extremely short half-life (T1/2=51.77 minutes) when administered in vivo. Accordingly, the present inventors formed a hypothesis that the alloferon peptide is capable of operating even when D-form peptides, which are not naturally present in the body, are prepared rather than L-form peptides which are easily degraded in vivo, based on the fact that the alloferon peptide of the present disclosure acts as a zinc chelator rather than acting by interacting with a specific protein in vivo, thereby acting as a kind of zinc homeostasis maintaining agent that keeps the extracellular concentration of zinc constant, prepared D-form alloferon, and performed the same experiment as that of using L-form alloferon. As a result, the present inventors found that D-form alloferon also has the same biological activity as L-form alloferon. In addition, since D-form alloferon is a material that is not metabolized in vivo, it was confirmed that in vivo stability was appreciably improved (see FIG. 1). In consideration of the characteristics of D-form alloferon, it is thought that, in order to prevent the alloferon from being degraded in the body, only some amino acids are substituted with D-form amino acids, or only four histidines participating in zinc chelating or only four neighboring amino acid residues including the histidines are substituted with D-form amino acids, or conversely, even if only the four histidines or the neighboring amino acid residues including the histidines (e.g., one amino acid before and after the histidine) are fixed as L-form amino acids and the remaining amino acids are substituted with D-form amino acids, the alloferon will exhibit similar stability and biological activity to the D-form alloferon in which all the amino acids are substituted with D-form amino acids.

The composition according to an embodiment of the present disclosure may include a pharmaceutically acceptable carrier, and may further include a pharmaceutically acceptable adjuvant, excipient, or diluent in addition to the carrier.

As used herein, the term “pharmaceutically acceptable” refers to a composition which is physiologically acceptable and typically doesn't cause an allergic response such as gastrointestinal disturbance and dizziness, or a similar response when administered to humans. Examples of the carrier, excipient, and diluent may include lactose, dextrose, sucrose, sorbitol, mannitol, xylitol, erythritol, maltitol, starch, acacia rubber, alginate, gelatin, calcium phosphate, calcium silicate, cellulose, methylcellulose, polyvinylpyrrolidone, hydroxybenzoate, talc, magnesium stearate, and mineral oil. Additionally, fillers, anti-coagulants, lubricants, humectants, fragrances, emulsifiers, preservatives, etc., may be additionally contained.

Additionally, the composition according to an embodiment of the present disclosure may be formulated using a method known in the art to allow the rapid release, sustained release or delayed release of an active ingredient when the composition is administered to a mammal. Examples of the formulation include powder, a granule, a tablet, an emulsion, a syrup, an aerosol, a soft or hard gelatin capsule, a sterile injectable solution, and a sterile powder form.

The composition according to one exemplary embodiment of the present disclosure may be administered by various routes such as oral administration and parenteral administration, for example, suppository, transdermal, intravenous, intraperitoneal, intramuscular, intralesional, nasal, or intravertebral administration, and may be administered using an implantation device for sustained release or continuous or repeated release. The administration frequency may be once or several times per day within a desired range and the administration period is not particularly limited.

The composition according to an embodiment of the present disclosure may be administered by general systemic administration or topical administration, for example, intramuscular injection or intravenous injection, but when the composition is provided as a composition including a polynucleotide or an expression vector including the polynucleotide, the composition may be most preferably injected using an electroporator. As the electroporator, an electroporator for injecting commercially available DNA drugs into the body, for example, Glinporator™ made by IGEA in Italy, CUY21EDIT made by JCBIO, Co., Ltd. in South Korea, and SP-4a made by Supertech, Ltd. in Switzerland.

With respect to an administration route of the composition according to an embodiment of the present disclosure, the composition may be administered via any general route as long as the composition may reach a target tissue. The administration route may include, but is not limited to, parenteral administration, for example, intraperitoneal, intravenous, intramuscular, subcutaneous, and intrasynovial administration.

The composition according to an embodiment of the present disclosure may be formulated in a suitable form together with a pharmaceutically acceptable carrier that is commonly used. Examples of the pharmaceutically acceptable carrier include carriers for parenteral administration such as water, suitable oil, a saline solution, aqueous glucose, and glycol, and a stabilizer and a preservative may be further contained. Examples of the suitable stabilizer include antioxidants such as sodium hydrogen sulfite, sodium sulfite, or ascorbic acid. Examples of the suitable preservative include benzalkonium chloride, methyl- or propyl-paraben, and chlorobutanol. Moreover, the composition according to the present disclosure may suitably contain a suspending agent, a solubilizer, a stabilizer, an isotonic agent, a preservative, an adsorption inhibitor, a surfactant, a diluent, an excipient, a pH adjuster, a painless agent, a buffer agent, an antioxidant, or the like if necessary, depending on the administration method or the formulation. The pharmaceutically acceptable carriers and formulations suitable for the present disclosure, including those exemplified above, are described in detail in the literature [Remington's Pharmaceutical Sciences, latest edition].

The dosage of the composition according to an embodiment of the present disclosure to a patient varies with many factors including the patient's height, body surface area, age, sex, general health conditions, a particular compound being administered, time and route of administration, and other drugs being administered concurrently. The therapeutically active alloferon or a polynucleotide encoding the alloferon may be administered in an amount of 100 ng/body weight (kg) to 10 mg/body weight (kg), more preferably 1 μg/kg (body weight) to 1 mg/kg (body weight), and most preferably 5-500 μg/kg (body weight), but the dosage may be adjusted in consideration of the aforementioned factors.

The pharmaceutical composition of the present disclosure may be administered in a therapeutically effective amount.

As used herein, the term “therapeutically effective amount” refers to an amount sufficient to treat the disease at a reasonable benefit/risk ratio applicable to medical treatment, and an effective dose level may be determined based on the factors including the kind of individual, severity of disease, age and sex of individual, activity of drug, sensitivity to drug, administration time, administration route, excretion rate, treatment duration, and drugs used concurrently, and other factors well known in the medical field. The pharmaceutical composition of the present disclosure may be administered at a dose of 0.1 mg/kg to 1 g/kg, and more preferably, 0.1 mg/kg to 500 mg/kg. Meanwhile, the dose may be appropriately adjusted according to the patient's age, sex, and condition.

The composition of the present disclosure may be administered orally or parenterally, and in the case of parenteral administration, it is possible to perform the administration via any route of systemic administration or topical administration. The systemic administration may be performed via intravenous injection, intraperitoneal injection, or intramuscular injection, and the topical administration may be performed via intracranial administration, intracerebrospinal administration, subcutaneous injection, or the like. In addition, in the case of a gene therapy in which a polynucleotide encoding alloferon or an expression vector including the same is administered, it may be administered using electroporation.

Hereinafter, the present disclosure will be described in more detail with reference to Examples. The present invention may, however, be embodied in different forms and should not be construed as limited to the embodiments set forth herein. Rather, these embodiments are provided so that this disclosure will be thorough and complete, and will fully convey the scope of the present invention to those skilled in the art to which the present invention belongs.

### Example 1: Peptide Preparation

1-1: Synthesis of L-Form Peptides

L-form peptides of alloferon (SEQ ID NO: 1) used in the present disclosure were synthesized in Peptron, Inc. (South Korea).

1-2: Synthesis of D-Form Peptides

D-form peptides having only D-form amino acids instead of L-form peptides used in the present disclosure were also synthesized in Peptron, Inc. (South Korea).

### Example 2: Protein Mass Analysis

The present inventors have thought that the mechanism of the alloferon peptide acts through the introduction process by endocytosis via multi-ligand receptors rather than physiological activity and the zinc binding ability acts as an important mechanism, and thus have confirmed the stability by substituting amino acids with the D-form amino acids. In vitro plasma stability analysis was performed in order to compare the stability of L-form peptides and D-form peptides of the alloferon in the plasma of mice. To this end, 500 to 600 μL of blood was obtained from ICR mice through retro-orbital bleeding, and then centrifuged at 2000 g at 4° C. for 20 minutes using a centrifuge, and only the supernatant was collected to obtain plasma. The 10 μM alloferon L-form and D-form peptides were put into 100 μL of plasma, respectively, and then allowed to stand at 37° C. for 0, 5, 10, 30, 60, and 90 minutes. Thereafter, 500 μL of cold methanol was added thereto to precipitate proteins, and the proteins were lyophilized, followed by liquid chromatography and protein mass spectrometry. As a result, it was found that the alloferon L-form peptides were degraded from an early time in plasma, whereas the D-form peptides were not degraded and remained stable over time (FIG. 1).

### Example 3: Nerve Cell Culture

The cerebral cortex nerve cells of the mice were obtained by extracting the cerebrum from ICR mouse embryos of 13-14 day gestation. 6-7 hemispheres per culture dish were inoculated on 24-well culture dishes or 6-well culture dishes (SPL Life Science, South Korea) coated with 0.01% poly-D-lysine (Sigma, USA) prepared in advance. A cell culture medium, in which Dulbecco's Modified Eagle's Medium (DMEM, Gibco, USA) to which 20 mM glucose and 38 mM sodium bicarbonate were added and which was further supplemented with 5% fetal bovine serum (Hyclone, USA), 5% horse serum (Gibco, USA), and 1% glutamine, was used, and the cell culture medium was incubated in a cell incubator, in which the conditions of 37° C. and 5% CO₂ were maintained, and was used in this experiment 10 to 12 days in vitro (DIV) after inoculation.

Three days after six hemispheres were inoculated into a 24-well culture dish to obtain pure nerve cells, 10 μM cytosine-D-arabinoside (ara-c, Sigma, USA) was added to the cell culture medium to inhibit the growth of non-nerve cells. Thereafter, it was used in this experiment 7 to 8 days in vitro (DIV) after inoculation.

In addition, in this study, H4 cell line (GL-H4) and 293T cell line in which the GFP-LC3 plasmid was permanently infected were used. As a cell culture medium, 10% fetal bovine serum (Hyclone, USA) and a mixture solution of antibiotics and antifungal agents (WellGene, South Korea) were added to Minimum Essential Medium (MEM, WellGene) and incubated in a cell incubator in which the conditions of 37° C. and 5% CO₂ were maintained.

### Example 4: Pre-Preparation Process and Induction of Cytotoxicity

In the pre-preparation process, 10-12 days after cell culture, the culture medium of the cerebral cortex nerve cells was changed to a minimum essential medium (MEM, Gibco, USA) without adding serum, and then was treated with N,N,N′,N′-tetrakis(2-pyridylmethyl)ethylene-diamine (TPEN, 2 μM, Sigma, USA), which is a zinc chelator, 50 μM chloroquine (CQ), 100 nM bafilomycin A-1 (Baf-A1), 500 μM methyl-beta-cyclodextrin (MβCD), and 2 μM chloropromazine (CP). Here, the MβCD is a caveolin-mediated endocytosis inhibitor, and CP is a clathrin-mediated endocytosis inhibitor. In addition, in the cell line experiments, the medium was changed to MEM according to the purpose of the experiments and was then treated with 40 μM chloroquine (CQ) and 100 nM bafilomycin A-1 (Baf-A1). During the pre-preparation process, the medium was treated with alloferon D-form peptides (0.2, 2, or 20 μM), free zinc (25 μM), TPEN (1 μM), and the like separately or together according to the purpose of each experiment.

### Example 5: Effect of Alloferon on Autophagic Flux Blocked by Chloroquine

In order to investigate the effect of the alloferon D-form peptides on the autophagic flux, the present inventors changed the medium to MEM in the H4 cell line (GL-H4) which expressed GFP-linked LC3, then inhibited autophagy using the alloferon D-form peptides (0.2, 2, or 20 μM) or chloroquine (CQ) which is an autophagy inhibitor, and then investigated the effect of the alloferon D-form peptides through fluorescence microscopic analysis and western blot analysis. Specifically, RIPA lysis buffer (50 mM Tris, pH 7.5, 150 mM NaCl, 1% NP-40, 0.5% deoxycholic acid, 0.1% SDS, and 5 mM EDTA) to which a protease inhibitor and a phosphatase inhibitor (2 μg/mL aprotinin, 2 μg/mL leupeptin, 1 μg/mL pepstatin A, 1 mM phenyl-methylsulfonyl fluoride (PMSF), 1 mM Na₃VO₄, 5 mM NaF, and 10 mM Na₄P₂O₇) was added into a 6-well culture dish at 150 μL per well to lyse the cells, and the obtained cell lysate was left at 4° C. for 25 minutes. Thereafter, the lysate was centrifuged at 10,000 rpm for 5 minutes, and then only the supernatant was taken to remove cell debris and obtain a protein extract. Protein quantification was performed using BCA protein assay kit (Pierce Biotechnology, USA). Then, 5× sample buffer (300 mM Tris, pH 6.8, 10% SDS, 50% glycerol, 0.1% BPB, 2.5% mercaptoethanol, and 100 mM DTT) was mixed with a quantified sample and denatured at 95° C. for 5 minutes to prepare the sample. Thereafter, the proteins separated according to sizes by electrophoresis using an 8% to 15% SDS-polyacrylamide gel were transferred to a polyvinylidene difluoride (PVDF) membrane (Millipore, USA). Then, the protein-transferred membrane was blocked for 1 hour by adding 3% skimmed milk or 3% BSA to TBST. The blocked membrane was reacted using anti-LC3-I antibodies (Novus Biologicals, LLC., USA), anti-LC3-II antibodies (Novus Biologicals, LLC., USA), and anti-p62 antibodies (MBL international corporation, USA), and the anti-Actin antibodies (Sigma, USA) were used as a loading control. Antibodies that respectively recognize specific proteins were added to a solution in which 1% BSA was dissolved in TBST. For all blots, secondary antibodies were diluted in 2% skimmed milk or 2% BSA at a ratio of 1:10,000, and reacted, and enhanced chemiluminescence (iNTRoN Biotechnology, South Korea) and bioimaging system (MF-Chemibis, Shimadzu Scientific Korea Corporation, South Korea) were used to identify protein signals.

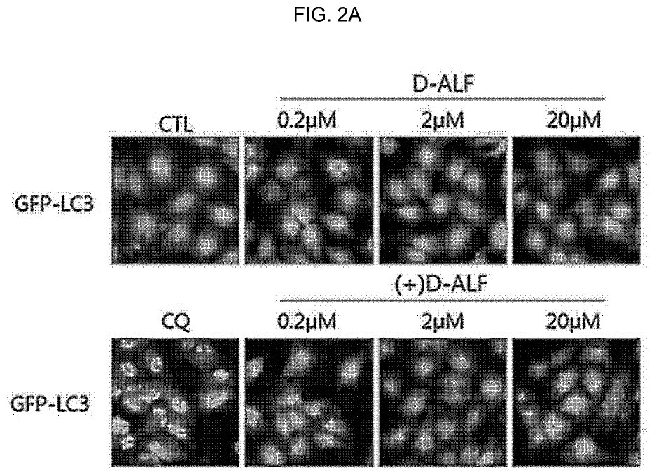

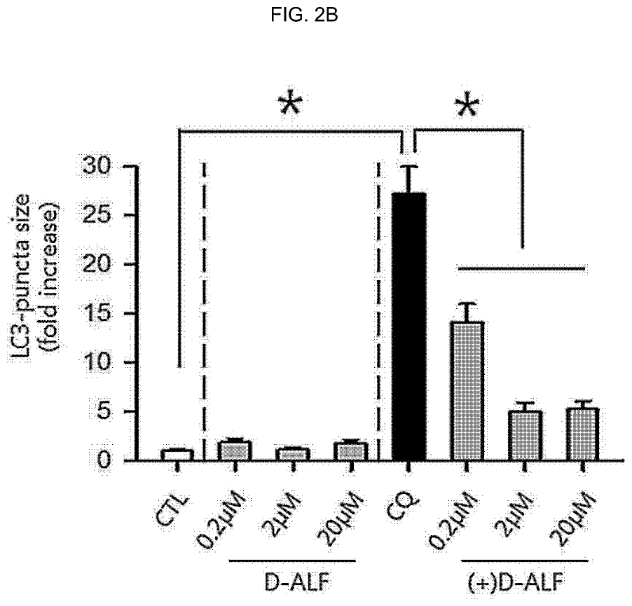

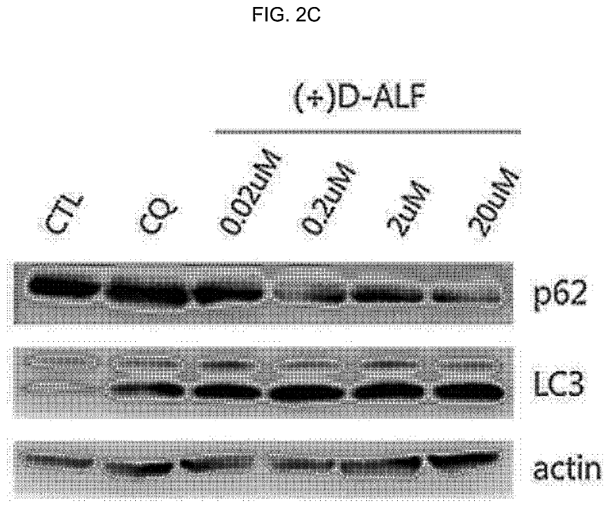

As a result, LC3 puncta shown in FIG. 2A are a phenomenon caused by the LC3-GFP proteins present in autophagosomes, which means that when the proteins are not degraded by lysosome, the entire autophagy process is inhibited and thus autophagosomes are accumulated. The inhibition of this autophagic flux also causes the accumulation of p62 proteins. When the cells were treated with the alloferon D-form peptide alone, it was expected that the LC3 puncta would have decreased compared to the control, but there was no significant difference, and when the GL-H4 cell line was treated with 50 μM CQ to inhibit the autophagic flux, the number and size of LC3 puncta were significantly increased, and this phenomenon was decreased in a concentration-dependent manner during the treatment of alloferon D-form peptides (0.2 μM, 2 μM, or 20 μM) (FIG. 2A). Similarly, when the size of the entire puncta was measured and quantified, the size of the puncta was decreased in a concentration-dependent manner during the treatment of alloferon D-form peptides and a significant decrease was shown from 0.2 μM (FIG. 2B). This means that the accumulated autophagosomes due to the blocked autophagy are destroyed by the alloferon, and it may be seen that alloferon alone does not affect the autophagy. In addition, p62 accumulated by CQ was reduced when treated with 0.02 μM alloferon D-form peptides, and LC3 conversion did not show any significant difference (FIG. 2C).

### Example 6: Lysotracker Staining

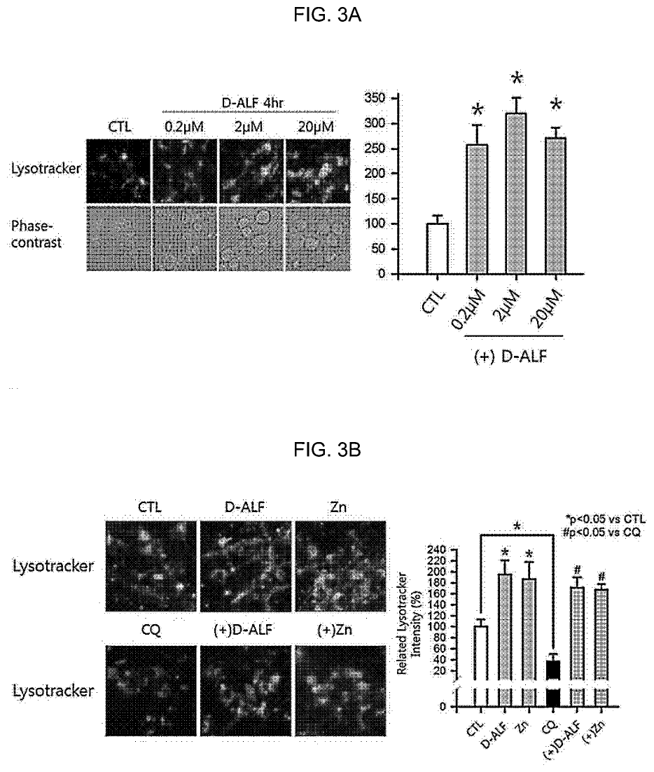

Since it was confirmed through FIG. 2 that alloferon may promote the autophagic flux, whether the alloferon D-form peptides are involved in pH change in the lysosome was confirmed through lysotracker staining. Specifically, after staining with 75 nM lysotracker (Thermofisher Scientific, Inc., USA) for 30 minutes, treatment with drugs (control, chloroquine, chloroquine+alloferon D-form peptides, chloroquine+zinc, alloferon D-form peptides, and zinc) was performed at a concentration described in Example 5 above and observation was performed by an optical microscope. It means that the stronger the fluorescence intensity, the lower the pH, and the weaker the fluorescence intensity, the higher the pH. It was confirmed that the pH in the lysosome was lowered by only the treatment with the alloferon D-form peptides compared to the control (FIG. 3A), and when treatment with 50 μM CQ was performed, the fluorescence intensity decreased compared to the control, and this was recovered by the treatment with the alloferon D-form peptides and zinc (FIG. 3B). This means that the pH in the lysosome raised by CQ is also lowered by alloferon as the case of treatment with zinc. That is, it could be considered that the alloferon D-form peptide lowers the lysosomal pH to improve the autophagic flux (FIG. 3).

### Example 7: Enzymatic Activity Analysis

The present inventors measured the activity of cathepsin B enzyme to confirm whether the alloferon D-form peptides have an effect on the enzymatic activity of cathepsin B, which is a representative protease in the lysosome. The cortical nerve cells were treated with 2 μM and 20 μM alloferon D-form peptides for 1 or 4 hours, respectively, and then staining was performed with 1× Magic Red substrate (Immunochemistry technologies, LLC., USA) and the stained cells were observed with a fluorescence microscope. It means that the stronger the fluorescence intensity, the stronger the enzymatic activity of cathepsin B, and the weaker the fluorescence intensity, the weaker the enzymatic activity. The degree of fluorescence activity of each experimental group was changed to the relative value and plotted.

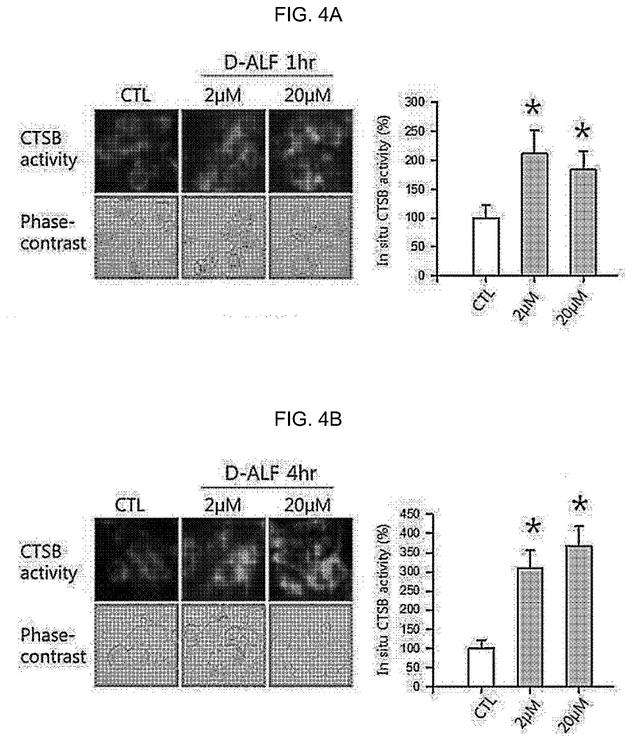

As a result, the activity of cathepsin B was increased by about 2 times when the treatment with the alloferon D-form peptides was performed for 1 hour (FIG. 4A), and the activity of cathepsin B was increased by about 3 times when the treatment was performed for 4 hours (FIG. 4B). Accordingly, it was confirmed that the alloferon D-form peptides increase the enzymatic activity of cathepsin.

### Example 8: Western Blot Analysis

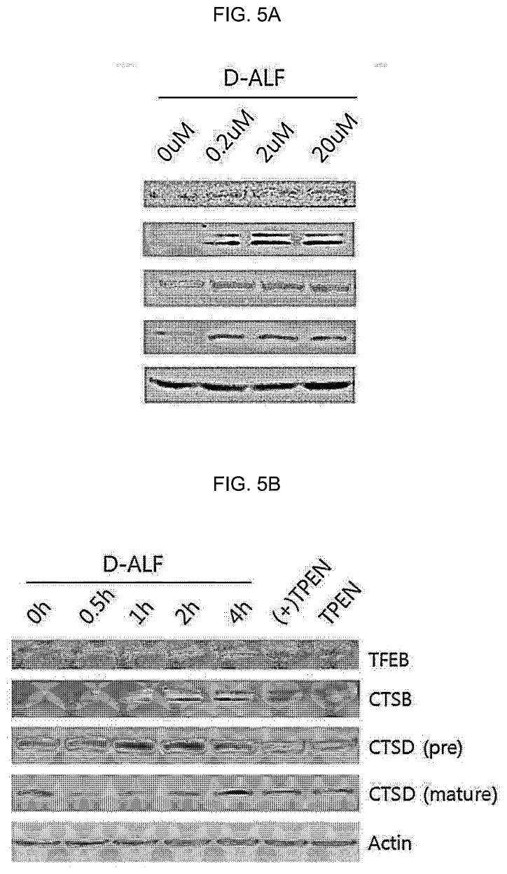

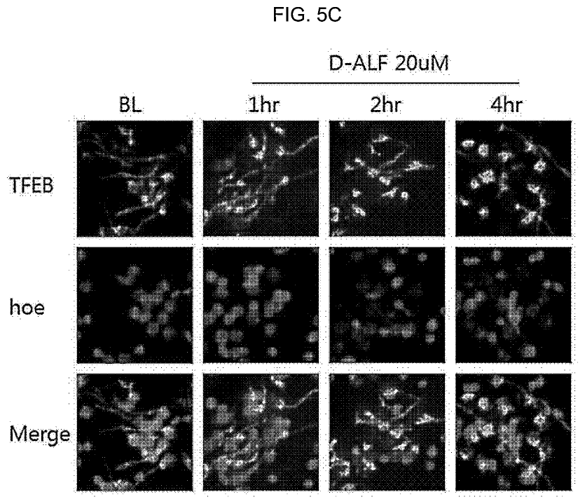

The present inventors had observed that the effect of enhancing the autophagic flux by the alloferon D-form peptides is achieved through the activity of cathepsin B and the decrease in the lysosomal pH, and thus, this time, the present inventors investigated through western blot analysis in order to find out whether there is a change in the amount of lysosomal protease expression. The western blot analysis was performed in the same manner as in Example 5, except that anti-TFEB-I antibodies (Bethyl laboratories, USA), anti-cathepsin B-antibodies (ABcam, UK), and anti-p62 antibodies (ABcam, UK) were used as the antibodies (FIG. 5).

As a result, the amounts of TFEB, cathepsin B, and cathepsin D were increased in a concentration-dependent manner during the treatment with the alloferon D-form peptides (FIG. 5A), and even in the case of the treatment with 20 μM alloferon D-form peptides, the amounts of TFEB, cathepsin B, and cathepsin D were gradually increased in a time-dependent manner. In addition, the amounts of TFEB, cathepsin B, and cathepsin D were reduced when TPEN, a zinc chelator, was supplied (FIG. 5B). That is, it means that the alloferon D-form peptides increase the amounts of TFEB, cathepsin B, and cathepsin D by increasing the zinc concentration. Here, TFEB is an important transcription factor for lysosome biosynthesis. Therefore, it was examined whether the transcription factor, TFEB, moved toward the nucleus or cell body after the alloferon treatment.

The immunocytochemical analysis for the position analysis of the TFEB was performed as follows:

Cerebral nerve cells seeded in a 24-well culture dish were treated with 20 μM alloferon D-form peptides for 1, 2, and 4 hours in accordance with the experimental conditions, and then the cells were fixed at room temperature with 4% PFA (pH 7.4) for 10 minutes, followed by washing twice with PBS. Then, the cells were reacted with 0.1% Triton X-100 (in PBS) at room temperature for 10 minutes and washed three times with PBS to perform cell permeation. Thereafter, 300 μL of 1% BSA (in PBST) was added per well, blocked at room temperature for 1 hour, and anti-TFEB-antibodies were diluted in the blocking solution at a ratio of 1:1000, 50 μL of the solution was added per well, and then, covered with a paraffin film having a square shape with the width and length of 1.2 cm to 3 cm, and reacted at 4° C. overnight. Then, after washing three times with PBS, the blocking solution in which secondary antibodies were diluted at a ratio of 1:100 was added thereto, covered with a paraffin film, wrapped with foil, reacted for 1 hour at room temperature, and washed three times with PBS for 5 minutes each. In order to observe the nuclei, a Hoechst solution in which the Hoechst dye was mixed at a ratio of 1:5,000 was added to PBS and stained for 15 minutes. Thereafter, it was washed once with PBS and photographed with a fluorescence microscope.

As a result, it was observed that the position of the TFEB moved from the neurites side to the cell body side having the nucleus in a time-dependent manner during the treatment with alloferon D-form peptides (FIG. 5C). That is, it may be seen that after the treatment with alloferon D-form peptides, the position of the TFEB moves toward the nucleus, and the expression of the TFEB increases and the nuclear movement increases the expressions of cathepsin B and cathepsin D.

### Example 9: Zinpyr-1 Staining

Subsequently, the present inventors measured the zinc concentration in the cells to determine what effect the treatment with the alloferon D-form peptides has on the zinc concentration in the lysosome. To this end, specifically, the cerebral cortical nerve cells were washed once with MEM, and then the MEM was replaced with MEM containing 5 μM zinpyr-1, an intracellular zinc marker (Cayman Chemical, USA, Ex=492 nm, Em=527 nm) and 75 nM lysotracker, and left at 37° C. for 30 minutes. Thereafter, it was washed with fresh MEM and treated with individual reagents (alloferon D-form peptides) according to the experimental conditions. Then, the cells were fixed with 4% PFA (pH 7.4) for 15 minutes, washed twice with PBS (Biowest, France), and photographed with a fluorescent microscope.

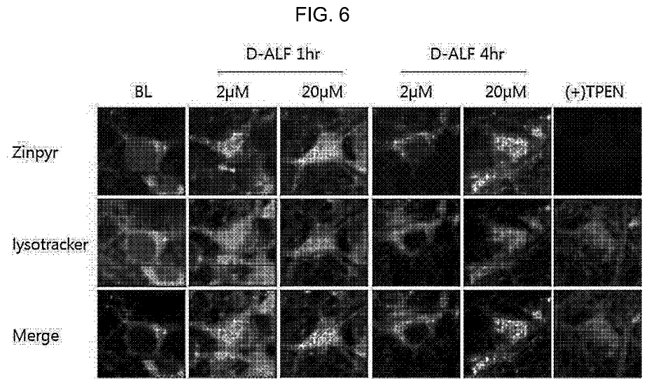

As a result, as shown in FIG. 6, the alloferon D-form peptides increased the zinc ions in the lysosome as compared to the control over time after the drug treatment (FIG. 6, increase in Zinpyr-1 fluorescence). In addition, the Zinpyr-1 fluorescence increased by the alloferon D-form peptides was reduced under the conditions in which the cells were treated with the alloferon D-form peptides in combination with TPEN, a strong zinc chelator. Accordingly, it was found that the alloferon D-form peptides substantially increase the zinc ions in the lysosome.

### Example 10: Analysis of Effect of Alloferon D-Form Peptides on Formation of Protein Aggregates after Overexpression of β-Amyloid Protein

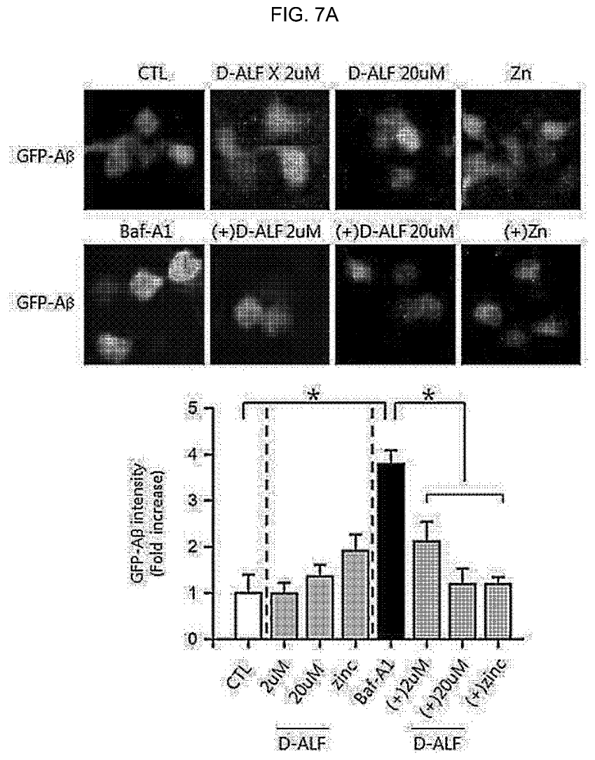

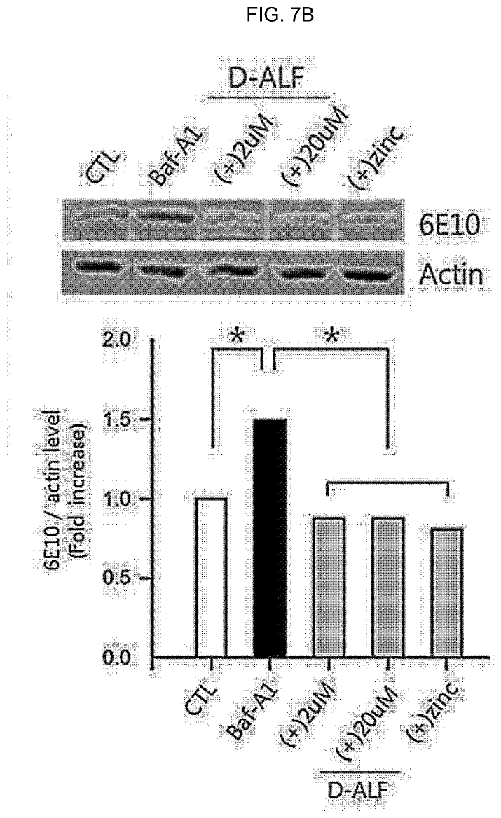

Subsequently, the present inventors investigated whether alloferon may dissolve the β-amyloid aggregates (Aβ aggregates) generated after the transient transfection of β-amyloid 1-42 (Aβ₁₋₄₂) into 293T cell lines. To this end, EGFP-tagged 1-42Aβ (pEGFP-C1-Abeta 1-42) DNA was transiently transfected into 293T cell lines using Lipofectamine2000. For cell observation, GFP signals were analyzed through a fluorescence microscope, and ImageJ was used for size and number analysis of GFP-positive aggregates. pEGFP-C1-Abeta 1-42 DNA was purchased from Addgene. Western blot analysis was performed in the same manner as in Example 5, except that anti-6E10-antibodies (Biolegend, USA) were used as the antibodies (FIG. 7).

As a result, there was no change in the protein aggregates when the cell line was treated with the alloferon D-form peptides or zinc compared to the control group, but when the protein aggregates were increased by Baf-A1, the protein aggregates were decreased when the alloferon D-form peptides or zinc was supplied (FIG. 7A), and even in the western blot analysis, the β-amyloid aggregates increased by Baf-A1 were decreased when the cell line was treated with the alloferon D-form peptides or zinc (FIG. 7B).

### Example 11: Analysis of Effect of Alloferon D-form Peptides on Formation of Protein Aggregates Generated by Mutant Huntington Protein

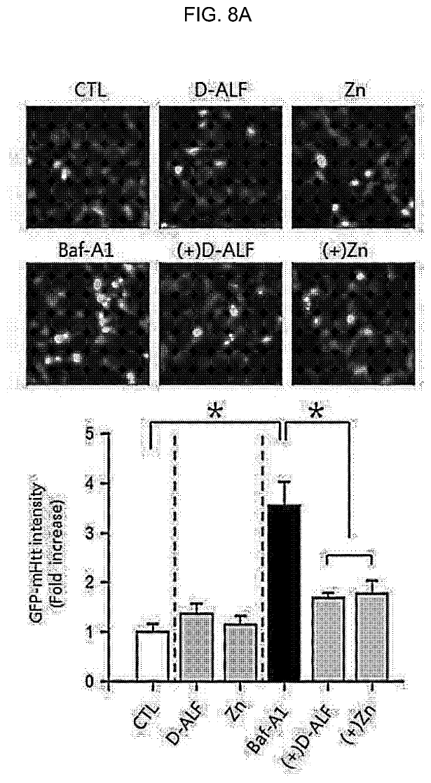

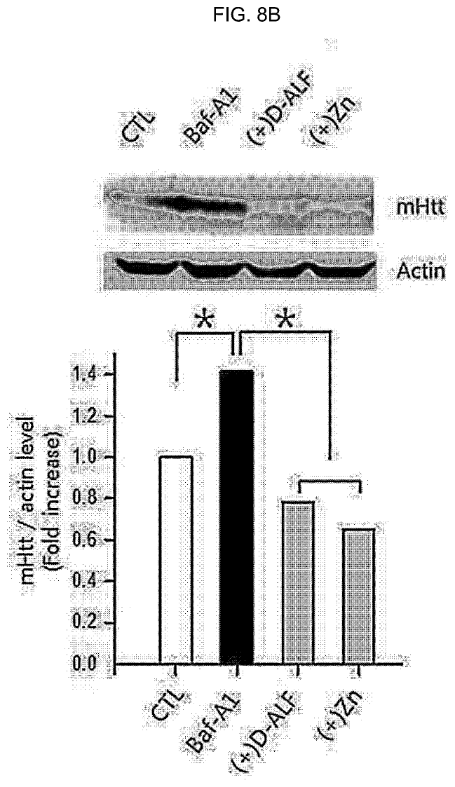

Subsequently, the present inventors investigated whether alloferon may dissolve Huntington protein aggregates (Htt aggregates) generated after the transient transfection of mutant Huntington protein (mt-Htt) into 293T cell lines. To this end, GFP-tagged Huntington Q74 (GFP-mHttQ74) DNA was transiently transfected into 293T cell lines using Lipofectamine2000. For cell observation, GFP signals were analyzed through a fluorescence microscope, and ImageJ was used for size and number analysis of GFP-positive aggregates (FIG. 8A). GFP-mHttQ74 DNA was provided by Dr. David C. Rubinsztein of the University of Cambridge, England (Park et al., Neurobiol. Dis. 42: 242-251, 2011). Western blot analysis was performed in the same manner as in Example 5, except that anti-poly-glutamine-antibodies (MilliporeSigma, USA) were used as the antibodies (FIG. 8B).

As a result, as shown in FIG. 8, when the treatment with the alloferon D-form peptide or zinc was performed, there was no change in the protein aggregates compared to the control, but when the protein aggregates were increased by Baf-A1, the protein aggregates were decreased when the alloferon D-form peptides or zinc was supplied. The alloferon D-form peptides may reduce Huntington protein aggregates as well as β-amyloid, and thus show the possibility of development as a drug that may be applied to degenerative neuronal diseases.

### Example 12: Investigation of Mechanism of Action of Alloferon

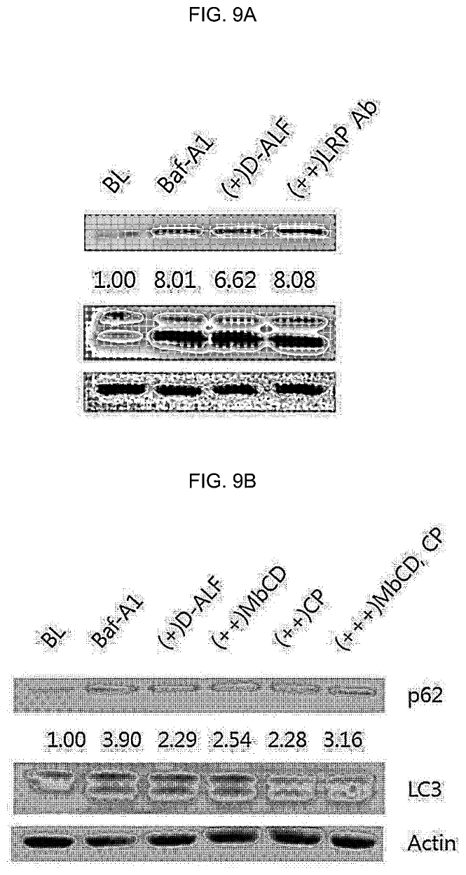

Since the protein cannot pass through the cell membrane, the present inventors thought that the alloferon D-form peptides would be introduced through an endocytosis process. Thus, the present inventors investigated whether the alloferon D-form peptides act through a receptor-mediated endocytosis process. Specifically, in order to examine whether the LRP1 receptor is involved, after 1 hour of pretreatment of Baf-A1 and antibodies that inhibit LRP1 receptor binding, the treatment of the alloferon D-form peptides were performed for 12 hours. As a result, it was found that the activation of the autophagic flux by the alloferon D-form peptides was eliminated by the LRP receptor inhibitor (FIG. 9A). In addition, it was observed that the autophagic flux blocked by Baf-A1 was activated by the alloferon D-form peptides, and the effect of the alloferon D-form peptides was not changed when MβCD, which inhibits the caveolin-mediated endocytosis process, or CP, which is the clathrin-mediated endocytosis inhibitor, was administered alone, but the activation effect of the alloferon D-form peptides was reduced and the accumulation of p62 proteins was increased when the MβCD and CP were administered concurrently (FIG. 9B). Accordingly, it was found that the receptor-mediated endocytosis process is important in regulating the autophagic flux by the alloferon D-form peptides.

### Example 13: Cytokine Analysis and Body Weight Change Observation

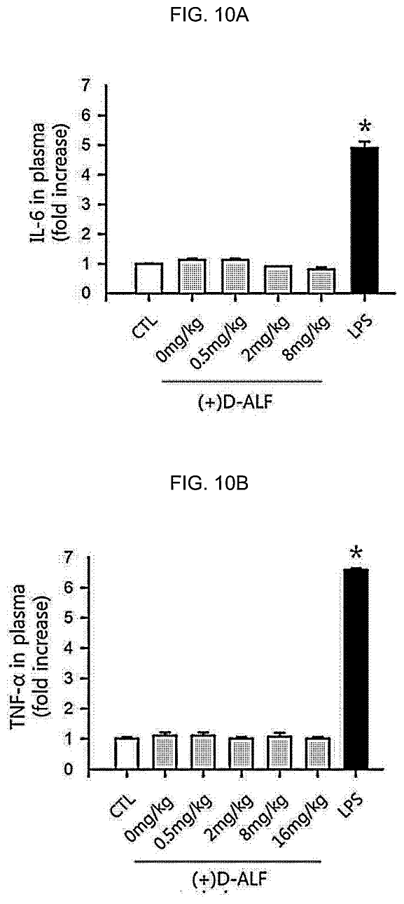

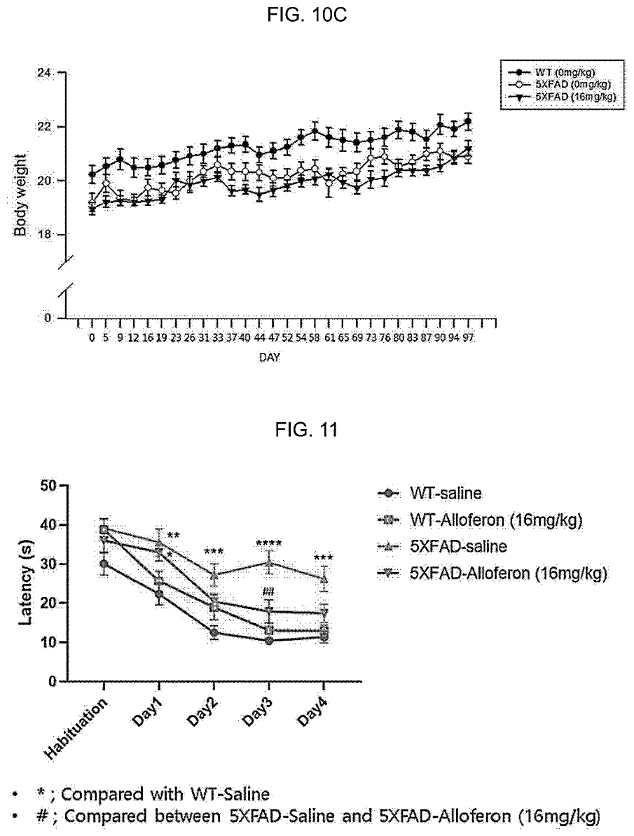

Since the present inventors confirmed the effect of improving autophagic flux in the alloferon D-form peptides from the experimental results, they tried to conduct a behavioral experiment by injecting the alloferon D-form peptides into dementia model animals. To this end, before conducting animal experiments, the alloferon D-form peptides were injected into mice, and then it was examined whether the immune response was induced. Specifically, the alloferon D-form peptides were intraperitoneally injected at 0.5, 2, 8, and 16 mg/kg, and after 1 hour, blood was collected from the retro-orbital plexus of the mice. The collected blood was centrifuged at 2,000 g at 4° C. for 20 minutes using a centrifuge, and then only the supernatant was taken to obtain plasma. Thereafter, an experiment was performed using TNF-α and IL-6 Elisa kit (RayBiotech, USA). Specifically, 100 μL of plasma sample was put into a 96-well microplate coated with each of anti-TNF-α and anti-IL-6 antibodies, and then left while shaking at room temperature for 2 hours and 30 minutes. Thereafter, the sample was discarded, washed 4 times with a wash buffer provided from the kit, and then 100 μL of the biotin-binding secondary antibodies was reacted for 1 hour. Then, it was washed 4 times with a washing buffer, 100 μL of streptavidin solution was added thereto, left while shaking at room temperature for 45 minutes, and washed again 4 times with a washing buffer. After 100 μL of TMB One-step substrate was added thereto and left for 30 minutes, 50 μL of a reaction termination solution was added thereto, and then 450 nm absorbance was measured by a spectrophotometer. As a result, as shown in FIG. 10, it was confirmed that the changes in TNF-α and IL-6 were not observed in all the conditions in which the alloferon D-form peptides were injected compared to the control (FIGS. 10A and 10B). In addition, it was observed that the amount of changes in the body weights of mice did not change during the 14-week intraperitoneal administration period (FIG. 10C). That is, it was found that even when the alloferon D-form peptides were injected into mice, it did not induce an immune response and a toxic response.

### Example 14: Water Maze Test

The present inventors observed that the alloferon D-form peptides degrade abnormal protein aggregates through the results of Examples 10 to 11, and thus observed whether memory improvement was shown in dementia model mice injected with the D-form peptides. To this end, after a mouse injected with a drug was put into a water maze, it was analyzed through camera recording whether the mouse swam in the water tank for 60 seconds to find an escape platform. This experiment was conducted 4 times a day for a total of 5 days including the acclimation period, and the average value was calculated by measuring the time required to find the escape platform 4 times a day. As a result, it was confirmed through statistical analysis that all the control injected with saline or alloferon D-form peptides gradually and efficiently found the escape platform at a similar time based on the memory through learning within 5 days, but it could be observed that dementia model mice injected with saline took a statistically significant long time to arrive at the escape platform compared to that of the control before the behavioral experiment was over, whereas dementia model mice injected with the alloferon D-form peptides efficiently found the escape platform in the tank at a level similar to that of the control by learning faster than the dementia model mice injected with saline. This may be thought that the memory and learning ability were improved by the alloferon D-form peptides (FIG. 11).

### Example 15: Analysis of Effect on Accumulation of β-Amyloid and Autophagy in Mouse Cerebrum

Subsequently, the present inventors confirmed whether alloferon intraperitoneally injected could dissolve the accumulation of β-amyloid in the brains of dementia model mice in which behavioral experiment was over. The brains of dementia model mice injected with saline or alloferon D-form peptides were extracted, and the cerebral cortices and hippocampi were obtained therefrom, and the tissues were pulverized and lysed with a RIPA dissolution buffer, and then western blot analysis and beta amyloid plaque staining were performed in the same manner as in Example 5.

The β-amyloid plaque staining was performed as follows:

The brains of dementia model mice injected with saline or alloferon D-form peptides were extracted, put into O.C.T compound (Sakura finetec, USA), lyophilized, and then brain tissue slices were obtained in a coronal section and attached to a 0.1% poly-L-lysine coated glass slide. The obtained brain tissue slices were washed with 70% ethanol for 1 minute, then washed again with 80% ethanol for 1 minute, and stained with 1% Thiflavin S solution (MilliporeSigma, USA) for 15 minutes. Then, the stained brain tissue slices were washed with 80% ethanol for 1 minute, washed again with 70% ethanol for 1 minute, and then washed twice with distilled water. The β-amyloid plaque was observed through a fluorescence microscope. The number of stained plaque per brain tissue slice was counted and quantified by a statistical program (Sigma plot).

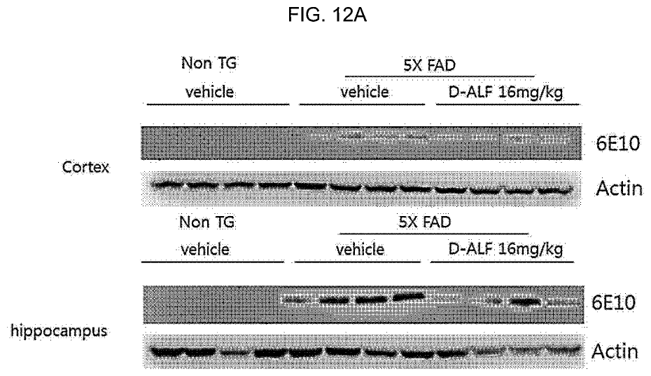

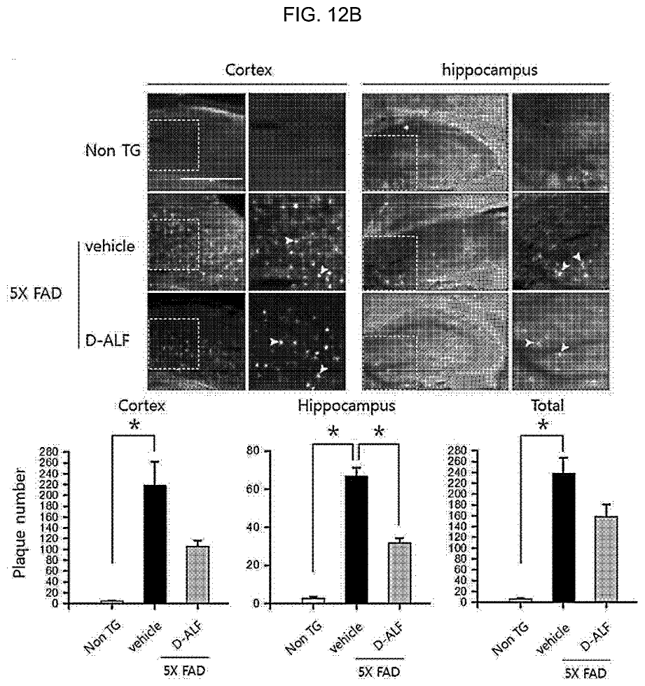

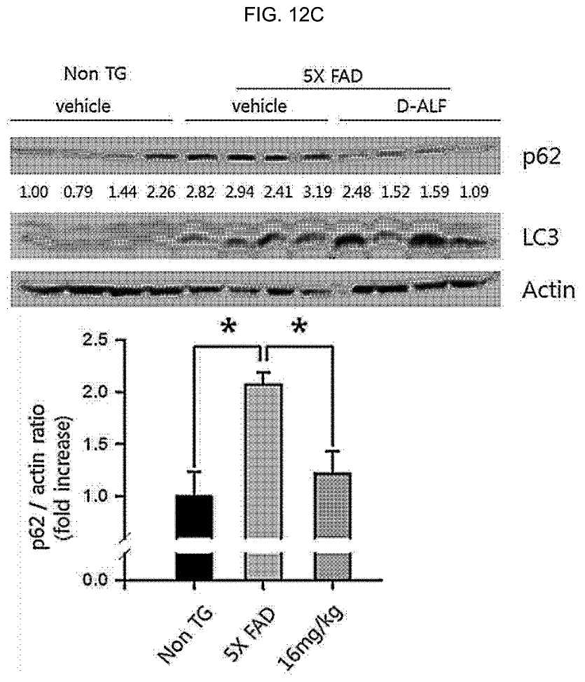

As a result, it was confirmed that the accumulation of β-amyloid oligomers was observed in the cerebral cortex and hippocampal tissues of the mice administered saline, and the accumulation of β-amyloid was decreased in the tissue sample injected with the alloferon D-form peptides (FIG. 12A), and it was observed that the number of plaque increased in the dementia model mice was decreased in the brains of mice injected with the alloferon D-form peptides compared to the control (FIG. 12B). In addition, it was confirmed that the accumulation of p62 protein was increased in the brains of mice in the dementia model group, whereas the accumulation of p62 protein was decreased in the group injected with the alloferon D-form peptides (FIG. 12C). Accordingly, it was found that the alloferon D-form peptides intraperitoneally injected into dementia model mice dissolve the accumulation of β-amyloid in cerebral cortex and hippocampal tissues.

It is possible to utilize the novel alloferon peptide and the method using the same of the present disclosure to effectively treat degenerative neuronal diseases such as Alzheimer's disease and Parkinson's disease by preventing apoptosis of nerve cells and regulating zinc homeostasis in cells. However, the scope of the present invention is not limited by such an effect.

The present invention is described with reference to the described examples, but the examples are merely illustrative. Therefore, it will be understood by those skilled in the art that various modifications and other equivalent embodiments can be made from the described embodiments. Hence, the real protective scope of the present invention shall be determined by the technical scope of the accompanying claims.

Although the novel alloferon peptide and the method using the same have been described with reference to the specific embodiments, they are not limited thereto. Therefore, it will be readily understood by those skilled in the art that various modifications and changes can be made thereto without departing from the spirit and scope of the present invention defined by the appended claims.

1. A method for treating a degenerative neuronal disease, comprising administering, to an individual suffering from a degenerative neuronal disease, a therapeutically effective amount of an alloferon peptide having an amino acid sequence of SEQ ID NO: 1.

2. The method of claim 1, wherein the alloferon peptide has typical L-form amino acids, include at least one D-form amino acid, or have amino acids, all of which are substituted with D-form amino acids.

3. The method of claim 1, wherein the degenerative neuronal disease has the formation of abnormal protein aggregates as a cause of the disease or a pathological phenomenon.

4. The method of claim 3, wherein the abnormal protein aggregates are formed by abnormal aggregation of α-synuclein, β-amyloid, Huntington protein, or tau protein.

5. The method of claim 3, wherein the degenerative neuronal disease having the formation of abnormal protein aggregates as a cause of the disease or a pathological phenomenon is Alzheimer's disease (AD), Parkinson's disease (PD), Huntington's disease (HD), chronic traumatic encephalopathy, Lytico-bodig disease, temporal lobe degeneration, corticobasal degeneration, progressive supranuclear palsy, or ganglioglioma.

6. The method of claim 1, wherein the alloferon peptide treats the degenerative neuronal disease by preventing cell death of nerve cells and regulating zinc homeostasis in cells.

7. A modified alloferon peptide in which at least one amino acid of an alloferon peptide having an amino acid sequence of SEQ ID NO: 1 is substituted with a D-form amino acid.

8. The modified alloferon peptide of claim 7, wherein the at least one amino acid is a histidine residue.

9. The modified alloferon peptide of claim 7, wherein four histidines are all substituted with D-form histidines.

10. The modified alloferon peptide of claim 7, wherein all the amino acids are substituted with D-form amino acids.

11. The modified alloferon peptide of claim 7, wherein all the amino acids except for the four histidines are substituted with D-form amino acids.

12. The modified alloferon peptide of claim 7, wherein all the amino acids except for the third and fourth amino acids are substituted with D-form amino acids.

13. The modified alloferon peptide of claim 7, wherein the third and fourth amino acids are substituted with D-form amino acids.

14. A composition comprising the modified alloferon peptide according to claim 7 as an active ingredient.

15. A method for treating a degenerative neuronal disease, comprising administering, to an individual, a therapeutically effective amount of the modified alloferon peptide according to claim 7.


SEQUENCE LISTING (1 sequence)

SEQ ID NO: 1 - protein, 13 aa, synthetic construct
  REGION 1..13: note = Alloferon peptide
HGVSGHGQHGVHG